(19) United States
(12) Patent Application Publication
(10) Pub. No.: US 2021/0236628 A1
(43) Pub. Date: Aug. 5, 2021

(54) SELF-EMULSIFYING SOLID ORAL DOSAGE FORMS OF ALLERGEN AND THEIR PREPARATION THEREOF

(71) Applicant: Jubilant Generics Limited, Noida (IN)

(72) Inventor: Indranil Nandi, Yardley, NJ (US)

(73) Assignee: Jubilant Generics Limited, Noida (IN)

(21) Appl. No.: 17/166,981

(22) Filed: Feb. 3, 2021

Related U.S. Application Data
(60) Provisional application No. 62/970,292, filed on Feb. 5, 2020.

(51) Int. Cl. A61K 39/36; A61K 9/00; A61K 9/50; A61K 39/35; A61K 47/38; A61K 47/26; A61K 47/32; A61K 47/02; A61K 47/36; A61K 47/14; A61K 47/10; A61K 9/48; A61K 9/20

(52) U.S. Cl. CPC A61K 39/36; A61K 9/2081; A61K 9/5089; A61K 39/35; A61K 47/38; A61K 47/26; A61K 47/32; A61K 47/02; A61K 47/36; A61K 47/14; A61K 47/10; A61K 9/5031; A61K 9/501; A61K 9/5047; A61K 9/5015; A61K 9/4808; A61K 9/0053

(57) ABSTRACT

The present invention relates to self-emulsifying pharmaceutical compositions in the field of allergen(s) and/or allergenic extract(s). Further within the scope of the invention is self-emulsifying solid oral pharmaceutical compositions comprising at least one allergen as an active agent and one or more pharmaceutically acceptable excipients. The present invention also relates to processes for preparing such compositions. These pharmaceutical compositions are useful in the treatment of various types of allergies such as drug allergy, food allergy, insect allergy, latex allergy, mold allergy, pet allergy, and pollen allergy.

### FIELD OF THE INVENTION

The present invention relates to self-emulsifying pharmaceutical compositions of allergen(s) and/or allergenic extract(s) and the method of manufacturing such compositions. In particular, but without restriction to the particular embodiments hereinafter described in accordance with the best mode of practice, the present invention provides self-emulsifying solid oral pharmaceutical compositions comprising at least one allergen as an active agent and one or more pharmaceutically acceptable excipients and process for preparing such compositions.

### BACKGROUND OF THE INVENTION

Allergy is a hypersensitivity disorder of the immune system, which is typically triggered by exposure to harmless environmental substances, known as allergens. There are various types of allergies, which may be seasonal or year-round allergies. However, some of the allergies may be life-long. Some examples of allergies include drug allergy, food allergy, insect allergy, latex allergy, mold allergy, pet allergy, and pollen allergy, which may not only pose substantial risks to an individual's health but also have a significant negative impact on an individual's quality of life.

As a treatment option for allergies, a number of known methods are available such as allergen immunotherapy, specific immunotherapy, or vaccination.

Conventional treatment options may include parenteral, oral immunotherapy (OIT), and sublingual immunotherapy (SLIT). However, multiple drawbacks and challenges are associated with these treatment options, such as the need for the patient to keep the product under the tongue for a definite time-period before swallowing or the need for the patient to mix the product with food or drink before swallowing. In addition, the amount of time that the allergen extract remains in the mouth is practically limited.

Conventionally, allergy vaccines to some extent, offer protective effect beyond the treatment period in contrast to symptomatic drug treatment, however, this approach encounters various limitations such as self-administration of uniform and precise dose by the patient, uniformity of drops, along with other formulation technical challenges such as the need to refrigerate the active ingredient and inclusion of preservatives in the formulation, to name a few.

U.S. Pat. No. 9,408,805 assigned to Alk Abello discloses fast-dispersing solid allergen dosage forms.

PCT Publication WO2007/051476 assigned to Alk Abello discloses the manufacturing of liquid vaccine formulation of allergen for oromucosal administration, for the treatment or prevention of allergy in a patient with a particular dosage regimen.

There exists an unmet need for improved methods of immunotherapy to treat various types of allergies such as drug allergy, food allergy, insect allergy, latex allergy, mold allergy, pet allergy, and pollen allergy.

Allergens and allergenic extracts are compounds of great potential and therapeutic benefit for immunization. Allergens and allergenic extracts, however, suffer from major drawbacks, which act as a barrier in its wider acceptance as a preferred method of choice.

Usually, allergens are protein in nature and have an extremely complex structure. Tight junctions in the gut wall resist their absorption due to which problems in oral bioavailability are encountered. The problem of poor oral bioavailability has also been attributed to a variety of other factors such as low stability, denaturation because of acidic pH, and enzymatic degradation. This may lead to loss of integrity, which may promote the inability to induce the appropriate immune response and sometimes may develop immune tolerance instead of protection.

These challenges pose major limitations in the development of oral pharmaceutical compositions of allergens and successful immunization. For the successful use of allergens and allergenic extracts as active agents in formulations, special treatment and formulation technology is required to overcome the above-mentioned challenges and ensure that the allergens remain stable during production, storage, and use.

The development of pharmaceutical compositions based on the lipid drug delivery systems offer an extensive variety of formulation approaches such as solutions, suspensions, solid dispersions, and self-emulsifying drug delivery systems (SEDDS). Pharmaceutical formulations, comprising oil or lipid, may exist as simple emulsions or self-emulsifying drug delivery systems (SEDDS). Depending upon the size, they are termed self-micro emulsifying drug delivery system (SMEDDS) or self-nanoemulsifying drug delivery system (SNEDDS). Self-emulsifying drug delivery systems usually comprise mixtures of natural or synthetic oils with surfactants and co-surfactants, which spontaneously emulsify when exposed to the gastrointestinal fluids or a liquid diluent to form an oil in-water or water in-oil emulsion or a micro- or nano-emulsion. These drug delivery systems offer various benefits, including, but not limited to, the ability to deliver active ingredients, which are sensitive towards enzymatic hydrolysis in GIT, improved solubilization, prolonged release of the active ingredient, reduced dosing frequency, reduced food effect, reduction in inter-subject and intra-subject variability, improved oral bioavailability and improved patient compliance. However, the development of pharmaceutical compositions based on a self-emulsifying drug delivery system is challenging from the formulation development perspective. A number of technical parameters need to be considered like selection of oil, surfactant, co-surfactant, solubility of the allergen or allergenic extract in different oils, oil-surfactant pairs, and cosolvents and their concentrations, temperature at which self emulsification occurs, and preparation of the phase diagram, to name a few.

PCT Publication WO95/08983 assigned to Gattefosse discloses a self-micro emulsifying drug delivery system comprising an NSAID as an active compound, lipophilic phase, surface-active agent, co-surfactant, hydrophilic phase that forms a micro-emulsion in situ when in contact with biological fluids.

U.S. Pat. No. 8,835,509 assigned to Arbro Pharmaceuticals discloses a self-emulsifying drug delivery system of curcuminoid comprising an oil phase, surfactant, and co-surfactant.

U.S. Pat. No. 6,221,391 assigned to Accucaps Industries discloses self-emulsifying solutions of Ibuprofen consisting of polyoxyethylene derivative of castor oil and polyvinylpyrrolidone, which is suitable for encapsulation into a soft gelatin capsule.

However, the above prior arts fail to offer the application of self-emulsifying drug delivery technology in the field of allergen or allergenic extracts.

Despite the existence of previously available therapies to treat various allergic diseases, the need still exists for a hands-on formulation technology which will better accommodate the specific limitations associated with the development of allergens in pharmaceutical compositions. Further, there is a need for new pharmaceutical compositions of allergens that are capable of self-emulsification and free from limitations of the prior arts such as degradation of allergens in the gastro-intestinal environment to realize allergen's therapeutic potential to treat various types of allergies.

Therefore, a need exists to develop self-emulsifying solid oral pharmaceutical compositions of allergen(s) and/or allergenic extract(s) with desirable technical attributes such as solubility, dispersibility, dissolution, stability, bioavailability, immunogenicity, and substantially free from hazardous amounts of allergen residues. Further, self-emulsifying pharmaceutical compositions as per the present invention can be manufactured by a simple, reproducible, and commercially viable process in the pharmaceutical laboratory as well as at an industrial scale.

### SUMMARY OF THE INVENTION

The present invention relates to self-emulsifying pharmaceutical compositions of allergen(s) and/or allergenic extract(s) and methods of manufacturing such compositions.

The present invention also relates to self-emulsifying solid oral pharmaceutical compositions comprising at least one allergen as an active agent and one or more pharmaceutically acceptable excipients and processes for preparing such compositions.

The present invention also relates to self-emulsifying solid oral pharmaceutical compositions comprising at least one allergen as an active agent and one or more pharmaceutically acceptable excipients, like diluent, binder, disintegrant, lubricant, solubilizer, gliclant, surfactant, co-surfactant, oil phase, carrier, inert sphere, seal coating polymer, enteric coating polymer, flavouring agent, taste masking agent, preservative, antioxidant, stabilizer, viscosity modifier, colouring agent, pH modifier, gelling agent, sweetener, vehicle, antacid, cryoprotectant and lyoprotectant.

The present invention further relates to self-emulsifying solid oral pharmaceutical compositions comprising at least one allergen as an active agent and one or more pharmaceutically acceptable excipients, which exhibit desired pharmaceutical technical attributes such as solubility, dispersibility, dissolution, stability, bioavailability, and immunogenicity and are substantially free from hazardous amounts of allergen residues.

The present invention also relates to the use of self-emulsifying solid oral pharmaceutical compositions of the present invention in the manufacture of a medicament for treating various types of allergies such as drug allergy, food allergy, insect allergy, latex allergy, mold allergy, pet allergy, and pollen allergy.

### DESCRIPTION OF THE INVENTION

The present invention can be more readily understood by reading the following detailed description of the invention and examples.

The present invention is based on a number of unforeseen outcomes, none of which could with a reasonable expectation of accomplishment be predicted in prior art. The present invention is based on the finding that it is feasible to use a self-emulsifying drug delivery system for the development of a solid oral dosage form containing at least one allergen and use of such compositions for the treatment of allergy. In particular, it has been revealed that it is certainly possible to attain the delivery of a sufficient effective amount of allergen to the immune system of the patient with the solid oral self-emulsifying drug delivery system of the present invention.

The term “pharmaceutical composition” as used herein refers to the composition, is intended to encompass a drug product comprising at least one allergen or at least one allergenic extract or any combination or mixture of at least one allergen and/or allergenic extract thereof, and the other inert ingredient(s) (pharmaceutically acceptable excipients). Such pharmaceutical compositions are synonymous with “formulation” and “dosage form”. Pharmaceutical compositions of the invention include, but are not limited to, powder, granules, sprinkles, concentrate, pellets, beads, powder, or granule formulation for compounding to produce a reconstituted liquid composition, tablets, capsules (immediate, modified, delayed, and controlled release) (hard and soft or liquid-filled soft gelatin capsules), pills, troches, sachets, microcapsules, mini-tablets, tablets in capsules and microspheres, matrix composition, liquid dosage forms such as solution, suspension, emulsion, micro emulsion, nano emulsion and the like. Preferably, the compositions as disclosed in the present invention include a capsule or tablet dosage form.

The term “excipient” refers to any ingredient that may be added to the formulation besides the active ingredient. The term “excipient” as used herein refers to a pharmacologically inactive component such as diluents, binders, disintegrants, lubricants, solubilizers, giidants, surfactants, co-surfactants, an oil phase, solid carriers, seal coating polymers, enteric coating polymers, antioxidants, viscosity modifiers, preservatives, stabilizers, pH modifiers, gelling agents, flavouring agents, taste masking agents, colouring agents, sweeteners, vehicles, antacids, cryoprotectant, lyoprotectant and the like and any combination thereof. The excipients that are useful in preparing a pharmaceutical composition are generally safe, non-toxic, and acceptable for veterinary as well as human use. Reference to an excipient includes at least one excipient in a suitable amount. Co-processed excipients are also covered under the scope of the present invention. Further, excipients may be in the form of powders or the form of a dispersion. Combinations of excipients performing the same function may also be used to achieve desired formulation characteristics.

As used in this specification, the singular forms “a”, “an”, and “the” include plural references unless the context clearly dictates otherwise. Thus, for example, a reference to “a process” includes one or more processes, and/or steps of the type described herein and/or which will become apparent to those persons skilled in the art upon reading this disclosure and so forth.

Any forms or tenses of one or more of these verbs “comprises,” “comprising,” “has,” “having,” “includes,” and “including” are also open-ended. For example, any method that “comprises,” “has” or “includes” one or more steps is not limited to possessing only those one or more steps and also covers other unlisted steps. “Optional” or “optionally” may be taken to mean that the subsequently described structure, event, or circumstance may or may not occur and that the description includes instances where the events occur and instance where it does not.

“Substantially free” as used herein refers to the self-emulsifying pharmaceutical compositions of allergen(s) and/or allergenic extract(s), which may contain acceptable limit or completely free from hazardous amounts of allergen residues.

As used herein, the term “therapeutic agent” means an agent utilized to treat, combat, ameliorate, prevent or improve an unwanted condition or allergy, or disease of a patient.

“Administering” a composition may be accomplished by oral administration and/or by other methods alone or in combination with other known techniques.

As used herein, the solid oral pharmaceutical composition of the present invention may be packaged in HDPE bottles or blister packs or calendar packs, or sachets. Packs may optionally contain desiccants and/or optionally instructions for preparation and use.

The term “controlled release” as used herein refers to a self-emulsifying pharmaceutical compositions in which release of the active agent is not immediate, i.e., with a “controlled release” formulation, the administration does not result in the immediate release of the drug. Generally, the term “controlled release” as used herein covers sustained release and delayed release. The term “delayed release” as used herein refers to self-emulsifying pharmaceutical compositions in which there is a time delay provided between oral administration and release of the drug therefrom. Preferred “delayed release” formulation is enteric coated compositions. The term “sustained release” as used herein refers to self-emulsifying pharmaceutical compositions that provide the release of a drug over an extended period of time.

The term “self emulsifying drug delivery” (SEDDS) as used herein refers to stable isotropic mixtures of active ingredient(s), oil(s), surfactant(s), and co-surfactant(s) and/or other pharmaceutical excipients. Self-emulsifying delivery systems are categorized as self-microemulsifying drug delivery systems (SMEDDS) and self-nanoemulsifying drug delivery systems (SNEDDS). SEDDS improves oral absorption of active ingredients by number of ways such as protection from the acidic environment, protection from enzymatic degradation, avoidance of first-pass metabolism, inhibition of gut efflux mechanism, improvement of solubility and permeability.

As used herein the term, “emulsion” refers to a binary pharmaceutical composition comprising an oil phase, drug, surfactant, and/or co-surfactant. Based on the constituents and the intended use, emulsions may be administered orally, topically, or parenterally. The emulsion formulations of the invention can be designed to be oil-in-water type (o/w), water-in-oil (w/o) type, multiple phasic and self-emulsifying drug delivery systems (SEDDS), which can be further categorized as self-micro emulsifying drug delivery systems (SMEDDS) or self-nanoemulsifying drug delivery systems (SNEDDS). As provided herein, self-micro or nano emulsifying droplets may have an average droplet size less than about 1000 nm, preferably less than 800 nm, preferably less than 500 nm, more preferably less than 300 nm, more preferably less than 200 nm, more preferably less than 100 nm and smaller ranges encompassed therein. The droplet size and zeta potential of the emulsion as per the present invention can be determined by Zetasizer or any other method known in the art.

The term “allergen or allergenic extract” as used herein refers to a type of antigen that can stimulate the immune response. In the present invention, the term allergen or allergenic extract(s) means the proteins, peptides, polysaccharides, or lipids molecules, which can induce allergic response(s) mediated by activation or stimulation of IgE antibodies. Allergenic extract can be an extract of biological allergen source material containing natural protein allergen that induces allergy. The allergen used herein may be in the form of a purified allergen, a modified allergen, an extract, a recombinant allergen, or mutant of a recombinant allergen. Allergen extracts refer to solutions or extracts of proteins, peptides, polysaccharides, lipid molecules, or glycoproteins, which are extracted from the allergen source. Preferably, allergen and allergenic extracts source include, but not limited to, pollen, weed, grass, epidermal, and inhalants (dog, cat, horse, guinea pig), insects, mite, food, mold, venoms, and combinations thereof. The allergen or combination of allergens used in the pharmaceutical composition according to the present invention can be in the form of allergenic extract or a combination thereof. Examples of natural allergens include pollen allergens (weed, grass, herb, tree pollen allergens), insect allergens (saliva, venom allergens, e.g. mite, cockroach, and midges allergens), animal hair, dander, and dandruff allergens (from e.g. dog, cat, horse, rat, mouse and the like), inhalation allergens (from fungi and molds) and food allergens (peanut, potato, egg and the like). Allergen(s) may constitute from about 0.001% to about 90% by weight of the composition, preferably 0.001% to 40%, more preferably 0.001% to 30%, more preferably 0.001% to 25%.

The term “lyophilization” or “lyophilizing” or “freeze-drying” as used herein refers to a process that permits removal of a solvent content by one or more sublimation processes. According to the present invention, lyophilization is carried out by freezing a sample in a first step and subsequently drying the sample in one or more steps via sublimation, optionally by reducing the surrounding pressure and/or by heating the sample so that the solvent sublimes directly from the solid phase to the gas phase. Lyophilized compositions have the advantages of better stability, enhanced efficacy, and improved patient compliance. The term “cryoprotectant” refers to a pharmaceutical excipient that is added to a composition in order to protect an active ingredient during the freezing cycle of a lyophilization process. The term “lyoprotectant” refers to a pharmaceutical excipient that is added to a formulation in order to protect an active ingredient during the drying stage of a lyophilization process.

Various tree pollen allergens/allergenic extracts include, but are not limited to, Acacia longifolia; Alnus rubra; Fraxinus americana; Fagus grandifolia; Paper, River/Red and White Birch; Boxelder, Hard Maple and Red Maple; Juniperus Ashei; Juniperus Virginiana; Populus deltoides; Cupressus arizonica; Taxodium distichum; Ulmus americana; Ulmus parvifolia; Eucalyptus globulus; Liquidambar styraciflua; Celtis occidentalis; Carya ovata; Tilia americana; Acer saccharum; Prosopis juliflora (glandulosa); Red and White Mulberry; Quercus rubra; Red, Virginia Live and White Oak; Olea europaea; Callistemon citrinus; Melaleuca quinquenervia; Cocos plumosa; Carya pecan (illinoensis); Lodgepole and Western Yellow Pine; Ligustrum vulgare; Elaeagnus angustifolia; Platanus occidentalis; Pecan, Maple BHR, Oak RVW, American Sycamore, Black Willow; White Ash, Birch PRW, Black Walnut, Common Cottonwood, American Elm; White Ash, River/Red Birch, Black Walnut, Common Cottonwood, American Elm, Shagbark Hickory, Hard Maple, Red Oak, American Sycamore, Black Willow; Juglans nigra; Salix nigra and the like.

Various weed pollen allergens/allergenic extracts include, but are not limited to, Amaranthus palmeri; Careless Weed and Rough Redroot Pigweed; Xanthium strumarium; Yellow Dock and Sheep Sorrel; Solidago canadensis; Kochia scoparia; Chenopodium album; Burweed, Povertyweed, and True Marshelder; Urtica dioica; Eupatorium capillifolium; Amaranthus retroflexus; Plantago lanceolata; Ambrosia trifida; Ambrosia artemisiifolia; Ambrosia psilostachya; Giant and Short Ragweed; Giant, Short, and Western Ragweed; Salsola kali; Artemisia vulgaris, Heterophylla (douglasiana); Atriplex canescens; Rumex acetosella; Common Cocklebur, Lamb's Quarters, Rough Redroot Pigweed, Dock/Sorrel.

Various grass pollen allergens/allergenic extracts include, but are not limited to, Paspalum notatum; Cynodon dactylon; Poa pratensis; Bromus inermis; Zea mays; Festuca elatior (pratensis); Kentucky Bluegrass, Orchard, Redtop, Timothy; Kentucky Bluegrass, Bermuda, Johnson, Redtop, Timothy; Sorghum halepense; Avena sativa; Dactylis glomerata; Agrostis gigantea (alba); Lolium perenne; Anthoxanthum odoratum; Phleum pratense.

Various epidermal and inhalant allergens/allergenic extracts include, but not limited to, Dog Hair and Dander (Mixed breeds); Feather Mix (Chicken, Duck, and Goose); Guinea Pig Hair and Dander; Cat Pelt; Cat hair; Cattle Hair and Dander; Horse Hair and Dander.

Various insect allergens/allergenic extracts include, but are not limited to, Fire Ant allergens including Solenopsis invicta, Solenopsis richteri, Solenopsis invicta, Solenopsis richteri, and the like; Cockroach allergens including Periplaneta americana; Blattella germanica.

Various mite allergens/allergenic extracts include, but are not limited to, Dermatophagoides pteronyssinus; and Dermatophagoides farina.

Various venom allergens/allergenic extracts include, but are not limited to, Honey Bee Venom (Apis mellifera); White-Faced Hornet Venom Protein (Dolichovespula maculata); Yellow Hornet Venom Protein (Dolichovespula arenaria); Wasp Venom Protein (Polistes spp.); and Yellow Jacket Venom Protein (Equal parts V. germanica, V. maculifrons, V. pensylvanica, V. vulgaris, and V. squamosa).

Various food allergens/allergenic extracts include, but are not limited to, Almond; Apple; Apricot; Banana; Barley, Whole Ground; Bean, Lima; Bean, Navy; Bean, String Green; Beef; Blueberry; Brazil Nut; Broccoli; Buckwheat; Cabbage; Cantaloupe/Muskmelon; Carrot; Cashew Nut; Catfish; Cauliflower; Celery; Cherry; Chicken Meat; Chocolate/Cacao Bean; Cinnamon; Clam; Coconut; Codfish; Coffee; Corn Food; Crab; Cucumber; Egg White, Chicken; Egg Whole, Chicken; Egg Yolk, Chicken; Filbert/Hazelnut Food; Flounder; Garlic; Grape; White Seedless; Fish (codfish, flounder, halibut, mackerel, tuna); Shellfish (clam, crab, oyster, scallops, shrimp); Hops Food; Lamb; Lemon; Lettuce; Lobster, Maine; Mackerel; Malt; Milk, Cow; Mushroom; Mustard Food; Nutmeg; Oat food; Olive, Green; Onion; Orange Food; Oyster; Pea, Green/English; Peach; Peanut (Ara h 1, Ara h 2, Ara h 3 and Ara h 6); Pear; Pecan Food; Pepper, Black; Pepper, Green; Pineapple; Pork; Potato, Sweet; Potato, White; Raspberry, Red; Rice Food; Rye Food; Saccharomyces (brewer's/baker's yeast); Salmon; Scallops; Sesame Seed; Shrimp; Soybean Food; Spinach; Squash, Yellow Summer; Strawberry; Tomato; Trout, Lake; Tuna; Turkey Meat; Vanilla; Walnut Food, Black; Walnut Food, English; Watermelon; and Whole Wheat.

Various mold allergens/allergenic extracts include, but are not limited to, Alternaria-Hormodendrum; Alternaria tenuis, Hormodendrum cladosporioides; Alternaria tenuis; Aspergillus fumigatus; Aspergillus niger; Botrytis cinerea; Candida albicans; Cephalosporium acremonium; Curvularia spicifera; Epicoccum nigrum; Epidermophyton floccosum; Fusarium vasinfectum; Helminthosporium interseminatum; Hormodendrum cladosporioides; Mucor racemosus; P. digitatum, expansum, roseum, notatum; Penicillium notatum; Phoma herbarum; Pullularia pullulans; Rhizopus nigricans; Stemphylium botryosum; Trichophyton; T. tonsurans, rubrum, mentagrophytes; Alternaria tenuis, Aspergillus (A. fumigatus, nidulans, niger, terreus); Hormodendrum cladosporioides; Penicillium (P. digitatum, expansum, notatum, roseum); Fusarium vasinfectum; Helminthosporium interseminatum; Mucor racemosus; Phoma herbarum; Pullularia pullulans; and Rhizopus nigricans.

There is no international accepted standardization method in the field of allergens and allergenic extracts. Many different units of extract strength exist such as SQ-Units (Standardized Quality Units), Standard Treatment Unit (STU), Standard Biological Unit (SBU), Sublinguale Eintheiten (SE), BAU (Biological Allergen Units), BU (Biological Units), Bioequivalent Allergen Unit (BAU), UM (Units of Mass), IU (International Units), Histamine Equivalent Unit (HEU), Standard Oral Unit (SOU), Therapeutic Unit (TU), Diagnostic Unit (DU), Index of concentration (IC) and IR (Index of Reactivity). The potency of allergens and allergenic extracts disclosed in the present invention can be represented with any of the methods known in the art. Preferably, the potency of allergens and allergenic extracts disclosed in the present invention can be represented with BAU (Biological Allergen Units).

Various methods are available for the characterisation of allergen such as Isoelectric Focusing (IEF), Crossed Immune Electrophoresis (CIE), Radial Immune Diffusion (RIE), Sodium Dodecyl Sulphate Polyacrylamide Gel Electrophoresis (SDS PAGE), Rocket Immuno Electrophoresis (RIE), Quantitative Immune Electrophoretic Techniques (QIE) and Enzyme Linked Immunosorbent Assays (ELISA). Further, the total activity of used allergen or allergenic extract may be performed by methods such as radio allergosorbent test, magic lite assay or ELISA based techniques, or any other method known in the art. Further immunization and disease management can be done by using diagnostic testing methods for the evaluation of allergic patients. A number of tests are available to perform this diagnosis, including, but not limited to, prick or scratch test (percutaneous test), blood test, or provocation test and intradermal testing (IDT) or any other method known in the art.

The term “stable” refers to the compositions of the present invention, wherein the loss in allergen content is less than 50% of the initial content after being stored for at least 1 day, preferably for at least 7 days, more preferably for at least 1 month, more preferably for at least 3 months. The stability of the solid oral composition may be evaluated at “long term” conditions 25° C./60% RH, at intermediate condition 30° C./65% RH, at “accelerated conditions” 40° C./75% RH or at refrigerated and/or freezed conditions −20° C. to 8° C., in the final container either measured as the loss in biological activity/potency or as the loss in the content of at least one allergen. Stability testing may be conducted according to the current guidelines by ICH and FDA. The loss may be measured by the ELISA method as described in the present invention or by any other method known in the art.

As used herein, the term “about” means±approximately 20% of the indicated value, such that “about 10 percent” indicates approximately 08 to 12 percent.

Unless otherwise stated the weight percentages expressed herein are based on the final weight of the composition or formulation.

The potency of the solid oral pharmaceutical compositions according to the present invention is from about 5-50,000 BAU/dosage form. Preferably, the potency is from 15-25,000 BAU/dosage form, preferably the potency is from about 15-18,000 BAU/dosage form, more preferably 650-3,500 BAU/dosage form.

In an embodiment, the present invention includes self-emulsifying pharmaceutical compositions of at least one or more allergen(s) and/or allergenic extract(s) and a method of manufacturing such compositions.

In another embodiment, the present invention includes self-emulsifying pharmaceutical compositions comprising at least one or more allergen as an active agent and one or more pharmaceutically acceptable excipients.

In another embodiment, the present invention includes self-emulsifying solid oral pharmaceutical compositions comprising at least one allergen as an active agent and one or more pharmaceutically acceptable excipients and process for preparing such compositions.

In another embodiment, the present invention includes self-emulsifying solid oral pharmaceutical compositions comprising at least one allergen as an active agent selected from the group consisting of tree pollen, weed pollen, grass pollen, epidermal, and inhalants (dog, cat, horse, guinea pig), insects, mite, food, mold and venoms and/or combinations thereof and one or more pharmaceutically acceptable excipients and process for preparing such compositions.

The present invention describes stable self-emulsifying solid oral pharmaceutical compositions comprising at least one allergen as an active agent, which exhibits desired pharmaceutical technical attributes like solubility, dispersibility, dissolution, stability, bioavailability, immunogenicity, and substantially free from hazardous amounts of allergen residues and process for preparing such compositions.

In an another embodiment of the invention, the present invention includes self-emulsifying solid oral pharmaceutical compositions comprising at least one allergen as an active agent and one or more pharmaceutically acceptable excipients, like diluent, binder, disintegrant, lubricant, solubilizer, glidant, surfactant, co-surfactant, oil phase, solid carrier, seal coating polymer, enteric coating polymer, flavouring agent, taste masking agent, preservative, antioxidant, stabilizer, viscosity modifier, colouring agent, pH modifier, gelling agent, sweetener, vehicle, antacid, cryoprotectant, lyoprotectant and the like and/or any combination thereof.

In another embodiment of the present invention, the self-emulsifying pharmaceutical compositions of the present invention are ideal for oral delivery systems. The compositions of the present invention are able to form in situ homogeneous, thermodynamically stable, uniform droplet sizes clear emulsion. The self-emulsifying compositions of the present invention can be administered in the form of solid dosage form, preferably as powder, capsule, mini-tablet, sprinkle and tablet dosage form.

In an embodiment, the particle size of the droplets as per the present invention is less than about 50 μm, preferably less than about 10 μm. In an embodiment, the particle size of the droplets as per the present invention is less than about 5 urn. In an embodiment, the particle size of the droplets as per the present invention is less than about 1000 nm, preferably less than about 800 nm. Preferably, the particle size of the droplets in the present invention are less than about 800 rim, 600 mu, 400 nm, more preferably less than 200 nm. The particle size can be measured by suitable techniques such as Laser light scattering (e.g. Malvern Light Scattering), dynamic light scattering (Zetasizer 3000, Malvern Inst.), Coulter counter microscopy, and any other technique known in the art.

In another embodiment of the present invention, the self-emulsifying solid oral pharmaceutical composition is a capsule or tablet or mini-tablet or powder or a sprinkle dosage form, which may be enteric coated to avoid acid and enzymatic degradation. The capsule shell can be gelatin-based or HPMC (hydroxypropyl methylcellulose or hypromellose) based or PVA (Polyvinyl acetate) based or cellulose ether film based or starch based. The capsule shell or tablet may be further processed to provide gastric protection by one or more suitable enteric polymer(s), enteric coating is impervious to an acidic environment, which dissolves within a pH range of about 4 to about 7.5. The size of the capsule may be selected to adequately contain the dose of the self-emulsifying pharmaceutical composition. The capsule sizes according to the present invention ranges from 5 to 000 including size 000, 00, 0, 1, 2, 3, 4, and 5. The composition according to the present invention is stable without separation and a composition that does not cause softening or deformation of the capsule shell.

In another embodiment of the present invention, the capsule shell is coated with an enteric polymer. Various useful enteric polymers according to the present invention include, but are not limited to, cellulose acetate trimellitiate (CAT) hydroxypropylmethyl cellulose phthalate (HPMCP), polyvinyl acetate phthalate (PVAP), cellulose acetate phthalate (CAP), shellac, and copolymers of methacrylic acid and ethyl acrylate.

In another embodiment of the present invention, the self-emulsifying solid oral pharmaceutical compositions may be prepared by conventional processes known to those of ordinary skill in the art, including, but not limited to, wet granulation, dry granulation such as slugging or compaction, lyophillization, or direct compression of the formulation into mini-tablets or tablets or filling into capsules or based on multi-unit particulate technology. Other formulation techniques are also contemplated within the scope of the present invention such as extrusion-spheronization, hot-melt extrusion, melt-granulation, freeze-drying, spray drying, mass extrusion and molding, solid dispersion (solvent evaporation, melting method, kneading method, co-grinding method, co-precipitation method, freeze-drying, spray-drying, coating, or adsorbing the drug onto carrier particles), multi-particulate based technology-pelletization and drug-ion exchange resin complexation.

In another embodiment of the invention, the self-emulsifying solid oral pharmaceutical compositions comprising at least one allergen as an active agent and one or more pharmaceutically acceptable excipients, prepared by the blending or mixing and compressed into a tablet or filled into a capsule. In the latter preparation, the acid susceptible allergen as an active agent is in the form of granule or multi particulate units layered with one or more enteric polymer.

In an embodiment, the self-emulsifying solid oral pharmaceutical compositions of the present invention comprise: a) at least one allergen in an amount of about 0.001-90% (w/w); b) oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients.

In an embodiment, the self-emulsifying solid oral pharmaceutical compositions of the present invention comprise: a) at least one allergen in an amount of about 0.001-90% (w/w); b) oil phase in an amount of about 0.01-70% (w/w), selected from the group consisting of propylene glycol esters, long chain fatty acids, edible oils, medium chain mono-, di-, or triglycerides, or a mixture thereof; c) surfactant in an amount of about 0.01-50% (w/w), selected from the group consisting of polyoxyethylene-sorbitan-fatty acid esters, polyoxyethylene products of hydrogenated vegetable oils, polyethoxylated castor oils or polyethoxylated hydrogenated castor oil, polyoxyethylene castor oil derivatives, polyethylene glycol caprylic/capric glycerides, polyethylene glycol glycerides or a combination thereof, d) co-surfactant in an amount of about 0-50% (w/w), selected from the group consisting of propylene glycol, diethylene glycol monoethyl ether, propylene glycol monocaprylate, glyceryl caprylate, ethanol, glyceryl caprylate, or a combination thereof; and e) optionally one or more pharmaceutically acceptable excipients.

In an embodiment, the self-emulsifying solid oral tablet composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) solid carrier in an amount of about 0-50% (w/w); and f) optionally one or more pharmaceutically acceptable excipients.

In another embodiment of the invention, the self-emulsifying solid oral tablet composition comprising at least one allergen as an active agent and one or more pharmaceutically acceptable excipients is prepared by the following steps: a) mixing at least one allergen to an oil phase, surfactant, co-surfactant to form a homogenous dispersion; b) adsorbing the homogenous dispersion obtained from step a) over suitable solid carrier, and c) compressing the adsorbed mixture obtained from step b) into a tablet dosage form.

In an embodiment, the self-emulsifying solid oral capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) solid carrier in an amount of about 0-50% (w/w); and f) optionally one or more pharmaceutically acceptable excipients.

In another embodiment of the invention, the self-emulsifying solid oral capsule composition comprising at least one allergen as an active agent and one or more pharmaceutically acceptable excipients, is prepared by the following steps: a) mixing at least one allergen to an oil phase, surfactant, co-surfactant to form a homogenous dispersion; b) adsorbing the homogenous dispersion obtained from step a) over suitable solid carrier; and c) encapsulating the adsorbed dispersion obtained from step b) in a capsule dosage form.

In an embodiment, the self-emulsifying solid oral tablet composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients.

In another embodiment of the invention, the self-emulsifying solid oral tablet composition comprising at least one allergen as an active agent and one or more pharmaceutically acceptable excipients is prepared by the following steps: a) mixing at least one allergen to an oil phase, surfactant, co-surfactant to form a homogenous dispersion, and b) compressing the homogenous dispersion obtained from step b) into the tablet dosage form.

In an embodiment, the self-emulsifying solid oral capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients.

In another embodiment of the invention, the self-emulsifying solid oral capsule composition comprising at least one allergen as an active agent and one or more pharmaceutically acceptable excipients is prepared by the following steps: a) mixing at least one allergen to an oil phase, surfactant, co-surfactant to form a homogenous dispersion, and b) encapsulating the homogenous dispersion obtained from step b) in a capsule dosage form.

In an embodiment, the self-emulsifying soft gelatin capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients.

In another embodiment of the invention, the self-emulsifying soft gelatin capsule composition comprising at least one allergen as an active agent and one or more pharmaceutically acceptable excipients is prepared by the following steps: a) mixing at least one allergen to an oil phase, surfactant, co-surfactant to form a homogenous liquid, and b) encapsulating the homogenous liquid obtained from step a) into a soft gelatin capsule.

In another embodiment of the invention, the self-emulsifying powder or granule pharmaceutical compositions comprise: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients, wherein the powder or granule compositions are further compressed into a tablet dosage form or encapsulated in a capsule dosage form, which will form in situ an emulsion upon contact with biological fluid after administration to the patient.

In another embodiment of the invention, the self-emulsifying powder or granule composition comprising at least one allergen as an active agent and one or more pharmaceutically acceptable excipients, is prepared by the following steps: a) mixing at least one allergen to an oil phase, surfactant, co-surfactant to form a homogenous dispersion; b) mixing the homogenous dispersion obtained from step a) with other excipients such as a binder, disintegrant, diluent; c) lubricating the blend of step b) with suitable lubricant; and d¹ compressing the lubricated blend into tablets or filling in capsules.

In another embodiment of the invention, the self-emulsifying solid oral tablet composition comprises: a) a core comprising at least one allergen, an oil phase, surfactant, and/or co-surfactant; b) a solid carrier; c) seal coating layer over the core; d) enteric coating layer over the seal coating; and e) optionally coated with an outer seal coating layer over the enteric coating layer.

In an embodiment, the self-emulsifying solid oral tablet composition of the present invention comprises: a) at least one allergen in an amount of about 0.001-90% (w/w); b) oil phase in an amount of about 0.01-70% (w/w); c) surfactant in an amount of about 0.01-50% (w/w); d) co-surfactant in an amount of about 0-50% (w/w); e) seal coating in an amount of about 0-20% (w/w); 0 enteric coating in an amount of about 0-45% (w/w); and g) optionally one or more pharmaceutically acceptable excipients.

In another embodiment of the invention, the self-emulsifying solid oral tablet composition comprising at least one allergen as an active agent and one or more pharmaceutically acceptable excipients, is prepared by the following steps: a) mixing at least one allergen, an oil phase, surfactant, and/or co-surfactant to prepare a core; b) adsorbing the prepared core from step a) over suitable solid carrier; c) seal coating layer over the core of step b); d) enteric coating layer over the seal coating; e) optionally coated with an outer seal coating layer over the enteric coating layer; and f) compressing into the tablet.

In another embodiment of the invention, the self-emulsifying solid oral capsule composition comprises: a) a core comprising at least one allergen, an oil phase, surfactant, and/or co-surfactant; b) a solid carrier; c) seal coating layer over the core; d) enteric coating layer over the seal coating; and e) optionally coated with an outer seal coating layer over the enteric coating layer.

In an embodiment, the self-emulsifying solid oral capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) seal coating in an amount of about 0-20% (w/w); f) enteric coating in an amount of about 0-45% (w/w), and g) optionally one or more pharmaceutically acceptable excipients.

In another embodiment of the invention, the self-emulsifying solid oral capsule composition comprising at least one allergen as an active agent and one or more pharmaceutically acceptable excipients, is prepared by the following steps: a) mixing at least one allergen, an oil phase, surfactant, and/or co-surfactant to prepare a core; b) adsorbing the prepared core from step a) over suitable solid carrier; c) seal coating layer over core of step b); d) enteric coating layer over the seal coat; e) optionally coated with an outer seal coating layer over the enteric coating layer; and f) encapsulating into a capsule.

In an embodiment, the self-emulsifying solid oral tablet composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) seal coating in an amount of about 0-20% (w/w); 0 enteric coating in an amount of about 0-45% (w/w); g) optionally one or more pharmaceutically acceptable excipients, and h) one or more enteric coating and/or seal coating layer.

In an embodiment, the self-emulsifying solid oral capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) seal coating in an amount of about 0-20% (w/w); 0 enteric coating in an amount of about 0-45% (w/w); g) optionally one or more pharmaceutically acceptable excipients, and h) one or more enteric coating and/or seal coating layer.

In an embodiment, the delayed-release self-emulsifying solid oral tablet composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) optionally a solid carrier in an amount of about 0-50% (w/w); f) optionally one or more pharmaceutically acceptable excipients, and g) one or more enteric coating and/or seal coating layer.

In an embodiment, the delayed-release self-emulsifying solid oral capsule composition of the present invention comprises: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); e) optionally a solid carrier in an amount of about 0-50% (w/w); f) optionally one or more pharmaceutically acceptable excipients, and g) one or more enteric coating and/or seal coating layer.

In another embodiment, there is provided a process for the preparation of delayed-release self-emulsifying solid oral pharmaceutical compositions, wherein the process comprises: a) mixing at least one allergen with an oil phase, surfactant, co-surfactant to form a homogenous dispersion, b) optionally heating the homogenous dispersion from step a), c) optionally adsorbing the homogenous dispersion from step b) over a solid carrier, d) blending the obtained dispersion with one or more pharmaceutically acceptable excipients, e) compressing the blend obtained from step d) into tablet dosage form or encapsulating in capsule dosage form and coating the tablets or capsule with an enteric polymer.

In an embodiment, the lyophilized self-emulsifying solid oral pharmaceutical compositions of the present invention comprise: a) at least one allergen in an amount of about 0.001-90% (w/w); b) an oil phase in an amount of about 0.01-70% (w/w); c) surfactant in an amount of about 0.01-50% (w/w); d) co-surfactant in an amount of about 0-50% (w/w); and e) optionally one or more pharmaceutically acceptable excipients.

In an embodiment, the self-emulsifying solid oral tablet or capsule composition of the present invention comprises: a) at least one allergen selected from the group comprising Phleum pretense, Lolium perenne, Ambrosia artemisiifolia, Periplaneta americana, Dermatophagoides farina, Apis mellifera, Peanut Allergen, and Aspergillus niger in an amount of about 0.001-25% (w/w); b) an oil phase selected from the group comprising propylene glycol dicaprylate and caprylic triglyceride in an amount of about 0.01-60% (w/w); c) surfactant selected from the group comprising polysorbate 80, labrasol, cremophor, and gelucire in an amount of about 0.01-50% (w/w); d) co-surfactant selected from the group comprising diethylene glycol monoethyl ether and propylene glycol monocaprylate in an amount of about 0.01-50% (w/w); e) diluent selected from the group comprising microcrystalline cellulose, lactose and mannitol in an amount of about 0.01-70% (w/w); f) binder selected from the group comprising hypromellose and povidone in an amount of about 0.01-10% (w/w); g) disintegrant selected from the group comprising crospovidone, croscarmellose sodium, and sodium starch glycolate in an amount of about 0.01-20% (w/w); h) glidant selected from the group comprising colloidal silicon di-oxide, starch, and talc in an amount of about 0.01-5% (w/w); i) lubricant selected from the group comprising magnesium stearate, sodium stearyl fumarate, and stearic acid in an amount of about 0.01-3% (w/w).

In an embodiment, the self-emulsifying solid oral tablet or capsule composition of the present invention comprises: a) at least one allergen selected from the group comprising Phleum pretense, Lolium perenne, Ambrosia artemisiifolia, Periplaneta americana, Dermatophagoides farina, Apis mellifera, Peanut Allergen, and Aspergillus niger in an amount of about 0.001-25% (w/w); b) an oil phase selected from the group comprising propylene glycol dicaprylate and caprylic triglyceride in an amount of about 0.01-60% (w/w); c) surfactant selected from the group comprising polysorbate 80, labrasol, cremophor, and gelucire in an amount of about 0.01-50% (w/w); d) co-surfactant selected from the group comprising diethylene glycol monoethyl ether and propylene glycol monocaprylate in an amount of about 0.01-50% (w/w); e) diluent selected from the group comprising microcrystalline cellulose, lactose and mannitol in an amount of about 0.01-70% (w/w); f) binder selected from the group comprising hypromellose and povidone in an amount of about 0.01-10% (w/w); g) disintegrant selected from the group comprising crospovidone, croscarmellose sodium, and sodium starch glycolate in an amount of about 0.01-20% (w/w); h) glidant selected from the group comprising colloidal silicon di-oxide, starch, and talc in an amount of about 0.01-5% (w/w); i) lubricant selected from the group comprising magnesium stearate, sodium stearyl fumarate, and stearic acid in an amount of about 0.01-3% (w/w); j) a seal coating over the core obtained in step i); k) an enteric coating over the seal coated core obtained in step j); 1) optional one or more seal and/or enteric coating over core obtained in step k).

In a further embodiment, the seal coating comprises up to 3% of polyethylene glycol, up to 3% of hypromellose, up to 1% of talc and purified water. In a further embodiment, the enteric coating comprises up to 20% of the methacrylic acid polymer, up to 3% of triethyl citrate, up to 2% of talc and purified water.

In another embodiment of the invention, the lyophilized self-emulsifying solid oral composition comprising at least one allergen as an active agent and one or more pharmaceutically acceptable excipients is prepared by the following steps: a) mixing at least one allergen to an oil phase, surfactant, co-surfactant to form a mixture; b) optionally blending the obtained mixture with one or more pharmaceutically acceptable excipients; c) lyophilizing the resultant mixture, and d) compressing the blend obtained from step c) into tablet dosage form or encapsulating in a capsule dosage form.

In accordance with still another embodiment of the present invention, there is provided a self-emulsifying solid oral pharmaceutical composition comprising at least one allergen as an active agent and one or more pharmaceutically acceptable excipients. The composition is prepared by simple mixing, blending, wet granulation, extrusion spheronization, dry granulation, spray drying, dry blending, lyophilization, multi-unit particulate technology (MUPs), or microencapsulation. Other formulation techniques are also contemplated within the scope of the present invention.

In another embodiment of the invention, the self-emulsifying compositions for oral administration comprising at least one allergen as an active agent and one or more pharmaceutically acceptable excipients, are prepared by heating and mixing the oil, surfactant, and co-surfactant. Optionally, the prepared mixture is then gently mixed for making the homogeneous formulation dispersion. The pre-concentrate is ready for encapsulation, which will form an in situ emulsion upon contact with biological fluid upon administration to a patient.

The compositions and processes disclosed herein can include the listed excipients or steps, can consist essentially of the listed excipients or steps, or consist of the listed excipients or steps.

In another embodiment of the invention, the present invention includes self-emulsifying solid oral pharmaceutical compositions comprising at least one allergen as an active agent and one or more pharmaceutically acceptable excipients, wherein the dose of the allergen and administration to the patient will vary depending on several factors including, but not limited to, the age of the patient, the severity of the condition of the patient, past medical history, among other factors, and will be determined by the physician in his sound discretion.

In another embodiment, the compositions according to the present invention can be a prescription or over the counter pharmaceutical composition.

In another embodiment, the present invention includes a self-emulsifying solid oral pharmaceutical composition comprising at least one allergen as an active agent and one or more pharmaceutically acceptable excipients, wherein the composition permits release at a pH range of about 4 to about 7.5.

In another embodiment of the invention, there is provided a self-emulsifying solid oral composition of the present invention, wherein the ratio of the weight of allergen to the weight of the oil phase is from 1:50 to 1:2, preferably, from 1:10 to 1:2. In another preferred embodiment, the ratio of allergen to the weight of the surfactant is from 1:50 to 1:2, preferably, from 1:10 to 1:2. In another preferred embodiment, the ratio of allergen to the weight of the co-surfactant is from 1:50 to 1:2, preferably, from 1:10 to 1:2. In another preferred embodiment, the ratio of oil to surfactant and/co-surfactant is from 1:40 to 1:2, preferably, from 1:10 to 1:2.

In a further embodiment, the present invention includes a method of compounding or prescribing the compositions of the present invention as per the instructions available with the compositions or can be determined by the physician at his sound discretion. The compounded compositions can be administered to the patient at least once daily, twice daily, or thrice daily. Preferably, the compositions of the present invention can be administered to a patient for at least one day, preferably at least two days, preferably at least one week, preferably at least two weeks, preferably at least three weeks, preferably at least one month, preferably at least two months, preferably at least three months, and for even longer based on the severity of the allergy.

In a further embodiment, the present invention includes a method of using the self-emulsifying solid oral pharmaceutical compositions of the present invention in the treatment of the various types of allergies such as drug allergy, food allergy, insect allergy, latex allergy, mold allergy, pet allergy, and pollen allergy.

The invention also features a method of treating an allergy by administering to an individual a composition as per the present invention in a synergistically effective amount to ameliorate the clinical course of the disease.

The term “oil” or “oil phase” as used herein refers to a non-aqueous phase of self-emulsifying drug delivery. Various useful oils according to the present invention include, but are not limited to, long-chain fatty acids, medium-chain mono-, di-, or triglycerides, propylene glycol esters or a mixture thereof, propylene glycol monocaprylate, edible oils such castor oil, arachis oil, cottonseed oil, soyabean oil, sesame oil, sweet orange oil, sun flower seed oil, peanut oil, canola oil rapeseed oil, and oleic acid, an essential oil such as peppermint oil, rosemary oil, orange oil, lemon oil, tea tree oil, wintergreen oil, lavender oil, ginger oil, nutmeg oil, fennel oil, eucalyptus oil, rosemary oil, or borage oil. In some embodiments, this list of essential oils further includes pomegranate seed oil, black cumin oil, rice germ oil, rice bran oil, krill oil, and green-lipped muscle oil, commercially available oils such as Oleyl Erucate, Propylene Glycol Dicaprate, Propylene Glycol Dicaprylocaprate, Propylene Glycol Dicaprylate/Dicaprate, Glyceryl Tricaprylate/Tricaprate, Caprylic/Capric Triglyceride, Caprylic/Capric/Linoleic Triglyceride, Caprylic/Capric/Succinic Triglyceride. The term ‘oil’ according to the present invention can be interchangeable with the term ‘lipid’ and/or “oil phase”. The oil phase may constitute from about 0.01% to about 90% by weight of the composition. In an embodiment, the oil phase according to the present invention is present in an amount of about 90% or less, e.g. 80% or less, 70% or less, 60% or less, 50% or less, 40% or less, 30% or less, 20% or less, 10% or less.

The compositions according to the present invention may further comprise one or more surfactants. Suitable surfactants according to the present invention are selected from ionic or non-ionic surfactants. Various useful surfactants include, but are not limited to, sodium lauryl sulphate, sodium cetyl stearyl sulphate or sodium dioctyl sulphosuccinate, lecithin, cetyl alcohol, stearyl alcohol, cetyl stearyl alcohol, cholesterol, sorbitan fatty acid esters such as sorbitan mono-oleate, polyoxyethylene sorbitan fatty acid esters such as polysorbate 80, polysorbate 20, polyoxyethylene fatty acid glycerides such as macrogol 1000 glycerol monostearate, polyoxyethylene fatty acid esters such as polyoxyl 40 stearate, polyoxyethylene fatty alcohol ethers such as polyoxyl 10 oleyl ether, glycerol fatty acid esters such as glycerol monostearate, polyethoxylated castor oils or polyethoxylated hydrogenated castor oil, polyoxyethylene-sorbitan-fatty acid esters, polyoxyethylene products of hydrogenated vegetable oils, polyoxyethylene castor oil derivatives or combination, lauroyl polyoxyl 32 glyceride or a polyoxyethyl hydroxyl stearate, a mixture of monoesters and diesters of 12-hydroxystearic acid and macrogols, commercially available surfactants includes, but not limited to, Cremophor® such as Cremophor® EL (PEG-35 castor oil), Cremophor® RH40 (PEG-40 hydrogenated castor oil), Cremophor® RH60 (PEG-60 hydrogenated castor oil), Labrasol® (PEG-8 Caprylic/Capric Glycerides), Polyethylene glycol (PEG) glycerides composed of mono-, di- and triglycerides and mono- and diesters of PEG, Gelucire® (Stearoyl polyoxylglycerides), Gelucire® 44/14, Gelucire® 50/13, Polysorbates, Pluronic® L-64 and L-127 (block copolymers based on ethylene oxide and propylene oxide), Triton™ X 100 (Polyethylene glycol tert-octylphenyl ether), Simulsol™ (polyoxyethylated products comprising Polyoxyethylated lauric alcohol, Polyoxyethylated cetostearyl alcohol, Polyoxyethylated stearic acid and the like) Nikkol™ HCO-50 (Polyoxyethylene (50) hydrogenated castor oil), Nikkol™ HCO-35 (Polyoxyethylene (35) hydrogenated castor oil), Nikkol™ HCO-40 (Polyoxyethylene (40) hydrogenated castor oil), Nikkol™ HCO-60 (Polyoxyethylene (60) hydrogenated castor oil), Span and Tweens®. The surfactant of the present invention may act as co-surfactant in the composition of the present invention. The term “surfactant” according to the present invention can be interchangeable with the term “emulsifier” or “solubilizer” or “co-solubilizer”. The amount of surfactant according to the present invention ranges from about 0% to about 80% by weight of the composition. In an embodiment, the surfactant according to the present invention is present in an amount of about 80% or less, e.g. 70% or less, 60% or less, 50% or less, 40% or less, 30% or less, 20% or less, 10% or less, 5% or less.

The term “co-surfactant” as used herein refers to the agent that enhances the emulsifying effect of the surfactant. The compositions according to the present invention may further comprise co-surfactant. Suitable co-surfactants according to the present invention include, but are not limited to, glycol ethers such as tetraglycol, a triglyceride or a derivative thereof, glycofurol (e.g., tetrahydrofurfuryl PEG ether), pyrrolidine derivatives such as N-methyl pyrrolidone (e.g., Pharmasolve®) and 2-pyrrolidone (e.g., Soluphor® P), short chain alcohols comprising from 1 to 6 carbons (e.g., ethanol), benzyl alcohol, alkane diols and trials (e.g., propylene glycol and its derivatives, glycerol, polyethylene glycols chain lengths such as PEG and PEG 400), bile salts, sodium deoxycholate, ethyl oleate, poloxamer, lecithin (phosphatidyl choline or lysophosphatidyl choline or like), phosphatidyl choline, Propylene Glycol, Caprylate or a phosphatidic acid derivative thereof (such as propylene glycol monocaprylate) commercially available products like TRANSCUTOL® (diethylene glycol monoethyl ether), CAPRYOL™ (Propylene glycol monocaprylate-Type I), CAPRYOL™ 90 (propylene glycol monocaprylate-Type II), CAPMULR (glyceryl caprylate), TETRA GLYCOL™ (tetrahydrofurfuryl diethylene glycol ether), LABRAFIL® (Polyglycosylglycerides), LUTROL® F68 (polaxomer 188), CARBITOL™ (diethylene glycol monoethyl ether), dextran, maltodextrin, lactose, mannitol, arabic gum, glycine, glucose, dextran 40 or mixtures and the like. The co-surfactants of the present invention may act as a solubilizer or co-solubilizer in the composition of the present invention. The term ‘co-surfactant’ according to the present invention can be interchangeable with the term “co-emulsifier”. The amount of co-surfactant according to the present invention ranges from about 0% to about 80% by weight of the composition. In an embodiment, the co-surfactant according to the present invention is present in an amount of about 80% or less, e.g. 70% or less, 60% or less, 50% or less, 40% or less, 30% or less, 20% or less, 10% or less, 5% or less, 2% or less.

Suitable solvents or vehicles according to the present invention include aqueous or organic solvents. Preferable solvents or vehicles include, but are not limited to, water, water for injection, ethanol, acidified ethanol, acetone, diacetone, polyols, polyethers, oils, esters, alkyl ketones, methylene chloride, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, acetaldehyde, acetone, benzene, carbon disulphide, carbon tetrachloride, 1,2 dichloroethane, dichloromethane, N,N-dimethylformamide, 1,4-dioxane, epichlorhydhn, ethyl acetate, ethanol, ethyl ether, ethylene glycol, 2-ethoxyethanol (acetate), formaldehyde, isopropanol, methanol, methyl n-butyl ketone, methyl ethyl ketone, 2-methoxyethanol (acetate), perchloroethylene, toluene, 1,1,1-trichloroethane, trichloroethylene, propylene glycol, triacetin, polyethylene glycol, tributyl citrate or triethyl citrate, glycerine, sorbitol, hexanetriol propylene carbonate, hexane glycol, sorbitans, alcohol ether, diethylene glycol monoethyl ether, 1,3-trimethyl-2-imidazolidone, phthalic acid esters, dibutyl sebacate, cetyl alcohol, polysorbates, dimethylisosorbide and mixtures thereof.

The compositions according to the present invention may further comprise one or more buffering agents. In an embodiment, the buffering agent according to the present invention is selected from a group consisting of acetic acid, glacial acetic acid, lactic acid, citric acid, phosphoric acid, carbonic acid, histidine, glycine, barbital, phthalic acid, adipic acid, ascorbic acid, maleic acid, amino acid, acetate, phosphate, tromethanol, succinic acid, tartaric acid, glutamic acid, glycolic acid, gluconic acid, gluconolactone, benzoic acid, aspartic acid, and salts (e.g., potassium, sodium, etc.) or combinations thereof. The amount of buffering agent according to the present invention ranges from about 0% to about 40% by weight of the composition. In an embodiment, the buffering agent according to the present invention is present in an amount of about 40% or less, e.g. 30% or less, 20% or less, 10% or less, 5% or less, 2% or less, 1% or less.

The compositions according to the present invention may further comprise gelling agents. Suitable examples of gelling agents according to the present invention include, but are not limited to, locust bean gum, xanthan gum, tragacanth, xylan, arabinogalactan, agar, gellan gum, guar gum, apricot gum (Prunus armeniaca, L.), carrageenan, pectin, acacia gum, dextran, Arabic gum, starch and starch derivatives, cellulose derivatives, such as microcrystalline cellulose, sodium carboxymethyl cellulose, methylcellulose, ethylcellulose, hydroxyethyl cellulose, hydroxypropyl cellulose, and hydroxypropyl methylcellulose, attapulgites, bentonites, dextrins, alginates, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof. The amount of gelling agent according to the present invention ranges from about 0% to about 40% by weight of the composition. In an embodiment, the gelling agents according to the present invention is present in an amount of about 40% or less, e.g. 30% or less, 20% or less, 10% or less, 5% or less, 2% or less, 1% or less.

The compositions according to the present invention may further comprise stabilizing agents. Suitable stabilizing agents according to the present invention include, but are not limited to, sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide), propyl gallate or malic acid, butylated hydroxytoluene, retinoids (e.g., retinol and retinyl palmitate), tocopherols (e.g., tocopherol acetate), tocotrienols, ubiquinone, erythrobic acid, benzoic acid, citric acid, sorbic acid, glucono-1,5-lactone (GDL), gluconic acid, lactic acid, calcium lactate, sodium erythorbate, sodium benzoate, sodium citrate, sodium sorbate, potassium sorbate, sodium gluconate, EDTA, disodium EDTA, trisodium EDTA, tetrasodium EDTA and mixtures thereof. The amount of stabilizing agent according to the present invention ranges from about 0% to about 40% by weight of the composition. In an embodiment, the stabilizing agent according to the present invention is present in an amount of about 40% or less, e.g. 30% or less, 20% or less, 10% or less, 5% or less, 2% or less, 1% or less.

The compositions according to the present invention may further comprise diluents or fillers. Diluents or fillers are substances, which usually provide bulk to the composition. Various useful fillers or diluents according to the present invention include, but not limited to, microcrystalline cellulose, calcium carbonate, calcium phosphate, dibasic anhydrous, calcium phosphate, dibasic dihydrate, calcium phosphate tribasic, calcium sulphate, cellulose powdered, cellulose acetate, compressible sugar, confectioner's sugar, dextrates, dextrose, fructose, lactitol, lactose, magnesium carbonate, magnesium oxide, maltodextrin, maltose, mannitol, polydextrose, simethicone, sodium alginate, sodium chloride, sorbitol, starch, pregelatinized starch, sucrose, trehalose, and xylitol, or mixtures thereof. The amount of diluent or filler according to the present invention ranges from about 0% to about 95% by weight of the composition. The term ‘diluent’ or ‘filler’ according to the present invention can be interchangeable with the term “carrier”. In an embodiment, the diluent according to the present invention is present in an amount of about 95% or less, e.g. 85% or less, 75% or less, 65% or less, 55% or less, 45% or less, 35% or less, 25% or less, 15% or less, 10% or less.

The compositions according to the present invention may further comprise binders. Binders impart cohesiveness to the formulation. Various useful binders according to the present invention include, but are not limited to, hypromellose, acacia, alginic acid, carbomer, sodium carboxymethylcellulose, dextrin, ethylcellulose, gelatin, glucose, guar gum, hydroxypropylcellulose, maltose, methylcellulose, povidone, copovidone, starch, polyvinyl alcohol or polyethylene oxide, or mixtures thereof. The amount of binder according to the present invention ranges from about 0% to about 30% by weight of the composition. In an embodiment, the binder according to the present invention is present in an amount of about 30% or less, e.g. 25% or less, 20% or less, 15% or less, 10% or less, 5% or less, 3% or less, 2% or less, 1% or less.

The compositions according to the present invention may further comprise glidants. Glidants improve the flowability and accuracy of dosing. Since the present invention relates to an oral pharmaceutical composition, it is imperative to use glidant(s) to achieve the desirable flowability of the active. Glidants used in the composition include, but not limited to, tribasic calcium phosphate, calcium silicate, cellulose powdered, colloidal silicon dioxide, magnesium silicate, magnesium trisilicate, starch, and talc or mixtures thereof. The amount of glidant according to the present invention ranges from about 0% to about 20% by weight of the composition. In an embodiment, the glidant according to the present invention is present in an amount of about 20% or less, e.g. 15% or less, 10% or less, 5% or less, 4% or less, 3% or less, 2% or less, 1% or less, 0.5 or less.

The compositions according to the present invention may further comprise disintegrants. Disintegrants according to the present invention are selected from the group consisting of crospovidone, modified starches, croscarmellose sodium, sodium starch glycolate, low substituted hydroxypropyl cellulose, and carboxymethylcellulose calcium. These disintegrants are also known as super-disintegrants. The amount of disintegrant according to the present invention ranges from about 0% to about 40% by weight of the composition. In an embodiment, the disintegrant according to the present invention is present in an amount of about 40% or less, e.g. 30% or less, 20% or less, 10% or less.

The compositions according to the present invention may further comprise lubricants. Suitable lubricants according to the present invention are selected from the group consisting of stearic acid, zinc stearate, sucrose stearate, sodium benzoate, hydrogenated vegetable oil, calcium stearate, adipic acid, glyceryl palmitostearate, glycerine monostearate, medium-chain triglycerides, sodium stearyl fumarate, glyceryl behenate, sodium lauryl sulphate, sodium stearyl fumarate, magnesium lauryl sulphate, magnesium stearate, polyethylene glycol. The amount of lubricant according to the present invention ranges from about 0% to about 10% by weight of the composition. In an embodiment, the lubricant according to the present invention is present in an amount of about 10% or less, e.g. 5% or less, 3% or less, 2% or less, 1% or less, 0.5% or less.

The term, “solid carrier” as used herein refers to porous, large surface area solid powder used to solidify the self-emulsifying system and form a solid self-emulsifying drug delivery system or formulation. The solid carriers according to the present invention include, but are not limited to, magnesium trisilicate, magnesium hydroxide, talcum, crospovidone, cross-linked sodium carboxymethyl cellulose, and cross-linked polymethyl methacrylate, calcium silicate, amorphous silicon dioxide, hydrophilic fumed silica, silicon dioxide, colloidal silicon dioxide, hydrophobic colloidal silica, magnesium aluminometasilicate, magnesium aluminometasilicate, magnesium stearate, starch, cellulose based derivatives, microcrystalline cellulose, methylcellulose, HPMC, lactose, croscarmellose sodium, anhydrous dibasic calcium phosphate, calcium carbonate, carbon nanotubes, carbon nanohorns, fullerene, charcoal, polyvinyl alcohol (PVA), sodium-CMC, dextran, zinc dioxide, dextrin, maltodextrin, cyclodextrin, hp-β-CD, and bamboo charcoal or mixtures thereof. The amount of solid carrier according to the present invention ranges from about 0% to about 90% by weight of the composition. In an embodiment, the solid carrier according to the present invention is present in an amount of about 90% or less, e.g. 80% or less, 70% or less, 60% or less, 50% or less, 40% or less, 30% or less, 20% or less, 10% or less.

The compositions according to the present invention may further comprise enteric polymers. Suitable enteric polymers according to the present invention selected from the group consisting of cellulose phthalates and derivatives thereof, cellulose acetate phthalate, cellulose acetate trimellitate (CAT), hydroxypropyl methylcellulose phthalate, cellulose acetate butyrate, hydroxypropyl methylcellulose acetate succinate, hydroxypropylcellulose acetate phthalate (HPCAP), polyvinyl acetate phthalate, cellulose acetate trimellitate, carboxymethylethylcellulose, shellac, ethylcellulose (EC), and polyvinyl phthalate acetate and methacrylic acid copolymers. Enteric polymers are commercially available from, amongst other companies, Degussa, Germany, under the brand names Eudragit® L 100-55 (powder form), Eudragit® L30 D-55 (30% aqueous dispersion of Eudragit® L 100-55), Eudragit L100 (powder form), and Eudragit® S 100 (powder form). The amount of enteric polymer according to the present invention ranges from about 0% to about 60% by weight of the composition. In an embodiment, the enteric polymer according to the present invention is present in an amount of about 60% or less, e.g. 50% or less, 40% or less, 30% or less, 20% or less, 10% or less, 5% or less.

A suitable inert core according to the present invention is selected from the group consisting of inert non-pareil sugar spheres, microcrystalline cellulose, corn starch, lactose, mannitol, glucose, fructose, maltose, erythritol, or sorbitol or mixture thereof.

The compositions according to the present invention may further comprise seal coating polymers. Suitable seal coating polymers according to the present invention are selected from the group consisting of ethylcellulose, polyethylene glycol, polyvinylpyrrolidone, hydroxypropylcellulose, hydroxypropyl methylcellulose, and methylcellulose, and a combination thereof. The amount of seal coating polymer according to the present invention ranges from about 0% to about 20% by weight of the composition. In an embodiment, the seal coating polymer according to the present invention is present in an amount of about 20% or less, e.g. 10% or less, 5% or less, 3% or less, 2% or less.

The compositions according to the present invention may further comprise plasticizers. Suitable plasticizers according to the present invention are selected from the group consisting of propylene glycol, triacetin, polyethylene glycol, tributyl citrate or triethyl citrate, triethyl citrate, glycerine, sorbitol, hexanetriol propylene carbonate, hexane glycol, sorbitans, tetrahydrofuryl alcohol ether, diethylene glycol monoethyl ether, 1,3-trimethyl-2-imidazolidone, phthalic acid esters, dibutyl sebacate, cetyl alcohol, polysorbates, and dimethylisosorbide. The amount of plasticizer according to the present invention ranges from about 0% to about 20% by weight of the composition. In an embodiment, the plasticizer according to the present invention is present in an amount of about 20% or less, e.g. 10% or less, 5% or less, 3% or less, 2% or less, 1% or less.

The compositions according to the present invention may further comprise alkaline agents. Alkaline agents may be used to provide a microenvironment of pH above 7 around the allergen and/or allergenic extract. Suitable alkaline agents according to the present invention include, but are not limited to, pharmacologically alkali metal, alkaline earth metal or metal salts of weak acids such as sodium carbonate which can be anhydrous or hydrous, calcium carbonate and magnesium carbonate, and the pharmacologically hydroxides and oxides of alkaline earth and earth metals such as magnesium hydroxide and magnesium oxide. The amount of alkaline agent according to the present invention ranges from about 0% to about 20% by weight of the composition. In an embodiment, the alkaline agent according to the present invention is present in an amount of about 20% or less, e.g. 10% or less, 5% or less, 3% or less, 2% or less, 1% or less, 0.5% or less.

The compositions according to the present invention may further comprise flavoring agents. Suitable flavoring agents according to the present invention include, but are not limited to, almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, vanilla, wintergreen, etc. or any other known flavoring agent known in the art. The amount of flavoring agent according to the present invention ranges from about 0% to about 30% by weight of the composition. In an embodiment, the flavoring agent according to the present invention is present in an amount of about 20% or less, e.g. 10% or less, 5% or less, 3% or less, 2% or less, 1% or less, 0.5% or less.

The compositions according to the present invention may further comprise solubilizers. Suitable solubilizers according to the present invention include, but not limited to, propylene glycol dicaprylate/dicaprate (Captex 200), propylene glycol monocaprylate (Capmul PG-8), propylene glycol caprylate/caprate (Labrafac PG), propylene glycol dicaprylate (Captex 100), propylene glycol diethylhexanoate, propylene glycol monolaurate (Capmul PG-12), glyceryl caprylate/caprate (Capmul MCM), glyceryl monocaprylate (Capmul MCMC-8, Imwitor 308), glyceryl monooleate (Capmul GMO), capric acid monoglyceride (Imwitor 312), PEG-6 corn oil (Labrafil M 2125), sorbitan monooleate (Span 80), sodium lauryl sulfate, sodium taurocholate, lecithin, lyso-lecithin, phosphatidyl glycerol, polyethylene glycol-phosphatidyl ethanolamines, cetyl trimethyl ammonium bromide, lauryl betaine, oleic acid, caprylic acid, capric acid, citric acid, tartaric acid, ascorbic acid, EDTA, cyclodextrin (various forms and derivatives thereof) and acetyl triethylcitrate, triethylcitrate, ethyl oleate, ethyl caprylate, triacetin, tetrahydrofurfuryl alcohol PEG ether (glycofurol), m-PEG, diethylene glycol monoethyl ether (Transcutol), diethylene glycol monobutyl ether, ethylene glycol monoethyl ether, ethanol, isopropanol, benzyl alcohol, ethylene glycol, propylene glycol, glycerol, sorbitol, mannitol, polyvinylalcohol, cellulose derivatives, polyethylene glycol (PEG 400 etc.), polypropylene glycol, POE-POP block polymers, pyrrolidones, N-alkylpyrrolidones, N-hydroxyalkylperrolidones, N-methylpyrrolidone, piperidones, N-alkylpiperidones, polyvinylpyrrolidones. In an embodiment, the solubilizer according to the present invention is present in an amount of about 30% or less, e.g. 20% or less, 10% or less, 5% or less, 3% or less, 2% or less, 1% or less, 0.5% or less.

The compositions according to the present invention may further comprise sweetening agents. Suitable sweetening agents according to the present invention include, but are not limited to, sugar or sugar alcohol such as sucrose, dextrose, sucralose, neotame, aspartame, acesulfame K, fructose, mannitol, invert sugar, saccharin sodium, xylitol, tagatose, maltitol, isomaltulose, hydrogenated isomaltulose, lactitol, sorbitol, erythritol, trehalose, maltodextrin, polydextrose, glycerin, inulin, maltol, alitame, sodium cyclamate, stevioside, thaumatin and the like. In an embodiment, the sweetening agent according to the present invention is present in an amount of about 30% or less, e.g. 20% or less, 10% or less, 5% or less, 3% or less, 2% or less, 1% or less, 0.5% or less.

The compositions according to the present invention may further comprise lyoprotectant. In an embodiment, the lyoprotectant according to the present invention is selected from a group consisting of sugars, monosaccharides or disaccharides, trehalose, mannitol, sucrose, lactose, maltose, or glucose, cellobiose galactose, raffinose, melezitose, maltodextrins, dextrans, dextrins, cellulose, starches and the like. Preferably, the lyoprotectant is selected from the group consisting of sucrose or a combination of sucrose and mannitol. The amount of lyoprotectant according to the present invention ranges from about 0% to about 90% by weight of the composition. In an embodiment, the lyoprotectant according to the present invention is present in an amount of about 80% or less, e.g. 60% or less, 40% or less, 20% or less, 10% or less, 5% or less.

The compositions according to the present invention may further comprise cryoprotectant. In an embodiment, the cryoprotectant according to the present invention is selected from a group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol and the like. The amount of cryoprotectant according to the present invention ranges from about 0% to about 90% by weight of the composition. In an embodiment, the cryoprotectant according to the present invention is present in an amount of about 80% or less, e.g. 60% or less, 40% or less, 20% or less, 10% or less, 5% or less.

The final formulations may be coated or uncoated. For coating, additional excipients such as film-forming polymers, plasticizers, anti-adherents, and opacifiers are used.

Having described the invention with reference to certain preferred embodiments, other embodiments will become apparent to one skilled in the art from consideration of the specification. The invention is further defined by reference to the following examples describing in detail methods for the preparation and testing of self-emulsifying pharmaceutical compositions. It will be apparent to those skilled in the art that many modifications, both to materials and methods, may be practiced without departing from the scope of the invention. The following examples are set out to illustrate the invention and do not limit the scope of the present invention.

### EXAMPLES

The following non-limiting examples are intended to further illustrate certain preferred embodiments of the invention. They are, however not intended to be limiting the scope of the present invention in any way.

EXAMPLES: Self-Emulsifying Drug Delivery (SEDDS) compositions prepared by using the quantitative formula as given in Table 1-5 (Quantity/Composition (% w/w)).

TABLE 1

| Ingredients | 1-Example (in % w/w) |
|---|---|
| Allergen(s) | 0.001-25 |
| Oil Phase | 0.01-70 |
| Surfactant | 0.01-60 |
| Co-Surfactant | 0-60 |

Optional Other Excipients

| | |
|---|---|
| Diluent | 0-70 |
| Binder | 0-10 |
| Disintegrant | 0-20 |
| Glidant | 0-5 |
| Lubricant | 0-3 |
| Solid Carrier | 0-50 |
| Buffering agent | 0-10 |
| Stabilizing agent | 0-10 |

### Brief Manufacturing Process:

a) Suitable allergen is added to the oil phase to form a dispersion; b) a suitable surfactant(s) is added to the dispersion formed in step a); c) a suitable co-surfactant(s) is added to the dispersion formed in step b); d) optionally, the homogenous dispersion obtained from step c) is adsorbed over a suitable solid carrier and optionally mixed with other pharmaceutical acceptable excipients; and e) the adsorbed mixture obtained is compressed into tablets or filled in capsule dosage forms.

TABLE 2

| Ingredients | 2-Example (in % w/w) |
|---|---|
| Allergen(s) | 0.001-25 |
| Oil Phase | 0.01-70 |
| Surfactant | 0.01-60 |
| Co-Surfactant | 0-60 |

Optional Other Excipients

| | |
|---|---|
| Diluent | 0-70 |
| Binder | 0-10 |
| Disintegrant | 0-20 |
| Glidant | 0-5 |
| Lubricant | 0-3 |
| Solid Carrier | 0-50 |
| Seal Coating | 0-15 |
| Enteric coating | 0-45 |
| Buffering agent | 0-10 |
| Stabilizing agent | 0-10 |
| Vehicle | q.s |

### Brief Manufacturing Process:

a) Suitable allergen is added to the oil phase, surfactant, co-surfactant, or other pharmaceutically acceptable excipients to form a homogenous dispersion; b) the homogenous dispersion obtained from step a) is adsorbed over a suitable solid carrier; c) the adsorbed mixture obtained from step b) is optionally mixed with other pharmaceutical acceptable excipients and encapsulated in a capsule or compressed into a tablet dosage form; d) the capsule or compressed tablet dosage form from step c) is coated with an enteric polymer.

TABLE 3

| Ingredients | 3 | 4 | 5 | 6 | 7 | 8 | 9 | 10 |
|---|---|---|---|---|---|---|---|---|
| Phleum pratense | 0.001-25 | — | — | — | — | — | — | — |
| Lolium perenne | — | 0.001-25 | — | — | — | — | — | — |
| Ambrosia artemisiifolia | — | — | 0.001-25 | — | — | — | — | — |
| Periplaneta americana | — | — | — | 0.001-25 | — | — | — | — |
| Dermatophagoides farina | — | — | — | — | 0.001-25 | — | — | — |
| Apis mellifera | — | — | — | — | — | 0.001-25 | — | — |
| Peanut Allergen | — | — | — | — | — | — | 0.001-25 | — |
| Aspergillus niger | — | — | — | — | — | — | — | 0.001-25 |
| Propylene Glycol Dicaprylate | 0.01-60 | — | 0.01-60 | 0.01-60 | 0.01-60 | — | 0.01-60 | 0.01-60 |
| Caprylic Triglyceride | — | 0.01-60 | — | — | — | 0.01-60 | — | — |
| Polysorbate 80 | 0.01-50 | — | — | — | — | — | — | — |
| Labrasol | — | 0.01-50 | — | — | 0.01-50 | — | 0.01-50 | — |
| Cremophor | — | — | 0.01-50 | — | — | — | — | — |
| Gelucire | — | — | — | 0.01-50 | — | 0.01-50 | — | 0.01-50 |
| Diethylene glycol monoethyl ether | 0.01-50 | 0.01-50 | 0.01-50 | 0.01-50 | 0.01-50 | 0.01-50 | — | — |
| Propylene Glycol Monocaprylate | — | — | — | — | — | — | 0.01-50 | 0.01-50 |
| Microcrystalline cellulose | 0.01-70 | — | 0.01-60 | — | — | 0.01-50 | — | 0.01-50 |
| Lactose | — | 0.01-70 | — | 0.01-50 | 0.01-50 | — | 0.01-50 | — |
| Hypromellose | 0.01-10 | — | 0.01-10 | — | 0.01-10 | 0.01-10 | — | 0.01-10 |
| Povidone | — | 0.01-10 | — | 0.01-10 | — | — | 0.01-10 | — |
| Crospovidone | 0.01-20 | — | — | — | — | 0.01-15 | — | — |
| Croscarmellose sodium | — | 0.01-15 | — | 0.01-15 | — | — | 0.01-15 | 0.01-15 |
| Sodium Starch Glycolate | — | — | 0.01-15 | — | 0.01-15 | — | — | — |
| Colloidal silicon di-oxide | 0.01-5 | 0.01-3 | 0.01-2 | 0.01-2 | 0.01-2 | 0.01-2 | 0.01-2 | 0.01-2 |
| Magnesium Stearate | 0.01-3 | 0.01-2 | 0.01-2 | 0.01-2 | 0.01-2 | 0.01-2 | 0.01-2 | 0.01-2 |

Manufacturing Process: a) Suitable allergen is added to the suitable oil phase (Propylene Glycol Dicaprylate or Caprylic Triglyceride) to form a dispersion; b) a suitable surfactant(s) (Polysorbate 80 or Labrasol or Cremophor or Gelucire) is added to the dispersion formed in step a); c) a suitable co-surfactant(s) (Diethylene glycol monoethyl ether or Propylene Glycol Monocaprylate) is added to the dispersion formed in step b); d) the homogenous dispersion obtained from step c) is adsorbed over a suitable solid carrier (microcrystalline cellulose or lactose) and optionally mixed with other pharmaceutically acceptable excipients; and e) the adsorbed mixture obtained is mixed with other excipients such as binder (hypromellose, povidone), disintegrant (crospovidone, croscarmellose sodium, sodium starch glycolate), glidant (colloidal silicon di-oxide), lubricant (magnesium stearate), and compressed into tablets or filled in capsule dosage forms.

TABLE 4

| Ingredients | 11 | 12 | 13 |
|---|---|---|---|
| Phleum pratense | 15 | — | — |
| Ambrosia artemisiifolia | — | 15 | |
| Periplaneta americana | — | — | 15 |
| Propylene Glycol Dicaprylate | 20 | 20 | 20 |
| Labrasol | 15 | — | 15 |
| Gelucire | — | 15 | — |
| Diethylene glycol monoethyl | 10 | 10 | 10 |
| ether | | | |
| Microcrystalline cellulose | 30 | — | 30 |
| Lactose | — | 30 | — |
| Hypromellose | 3 | — | 3 |
| Povidone | — | 3 | — |
| Crospovidone | 5 | — | — |
| Croscarmellose sodium | — | 5 | — |
| Sodium Starch Glycolate | — | — | 5 |
| Colloidal silicon di-oxide | 1 | 1 | 1 |
| Magnesium Stearate | 1 | 1 | 1 |

Manufacturing Process: As described in Table 3.

TABLE 5

| Ingredients | 14 | 15 | 16 |
|---|---|---|---|
| Phleum pratense | 15 | — | — |
| Ambrosia artemisiifolia | — | 15 | |
| Periplaneta americana | — | — | 15 |
| Propylene Glycol Dicaprylate | 10 | 10 | 10 |
| Labrasol | 10 | — | 10 |
| Gelucire | — | 10 | — |
| Diethylene glycol monoethyl | 10 | 10 | 10 |
| ether | | | |
| Microcrystalline cellulose | 25 | — | 25 |
| Lactose | — | 25 | — |
| Hypromellose | 2.8 | — | 2.8 |
| Povidone | — | 2.8 | — |
| Crospovidone | 5 | — | — |
| Croscarmellose sodium | — | 5 | — |
| Sodium Starch Glycolate | — | — | 5 |
| Colloidal silicon di-oxide | 1 | 1 | 1 |
| Magnesium Stearate | 1 | 1 | 1 |

Seal Coating

| | | | |
|---|---|---|---|
| Poly Ethylene Glycol | 1.3 | 1.3 | 1.3 |
| Hypromellose | 1.3 | 1.3 | 1.3 |
| Talc | 0.3 | 0.3 | 0.3 |
| Purified Water | q.s. | q.s. | q.s. |

Enteric Coating

| | | | |
|---|---|---|---|
| Eudragit | 13 | 13 | 13 |
| Talc | 1.3 | 1.3 | 1.3 |
| Triethyl Citrate | 3 | 3 | 3 |
| Purified Water | q.s. | q.s. | q.s. |

Manufacturing Process: a) Suitable allergen is added to the suitable oil phase (Propylene Glycol Dicaprylate) to form a dispersion; b) a suitable surfactant(s) (Labrasol or Gelucire) is added to the dispersion formed in step a); c) a suitable co-surfactant(s) (Diethylene glycol monoethyl ether) is added to the dispersion formed in step b); d) the homogenous dispersion obtained from step c) is adsorbed over a suitable solid carrier (microcrystalline cellulose or lactose) and optionally mixed with other pharmaceutically acceptable excipients; and e) the adsorbed mixture obtained is mixed with other excipients such as binder (hypromellose, povidone), disintegrant (crospovidone, croscarmellose sodium, sodium starch glycolate), glidant (colloidal silicon di-oxide), lubricant (magnesium stearate), f) optionally, the blend is seal coated followed by the enteric coating, g) and compressed into tablets or filled in capsule dosage forms.

The self-emulsifying solid oral pharmaceutical compositions comprising as per the present invention exhibits desired pharmaceutical technical attributes such as solubility, dispersibility, dissolution, stability, bioavailability, and immunogenicity.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of the invention.

What is claimed:

1. A self-emulsifying pharmaceutical composition for oral administration comprising:
a) at least one or more allergens as an active agent; and
b) one or more pharmaceutically acceptable excipients.

2. The pharmaceutical composition according to claim 1, wherein:
a) the at least one or more allergens is present in an amount of about 0.001-90% (w/w); and
b) the one or more pharmaceutically acceptable excipients comprise:
an oil phase in an amount of about 0.01-70% (w/w);
one or more surfactants in an amount of about 0.01-50% (w/w);
one or more co-surfactants in an amount of about 0-50% (w/w); and
optionally one or more pharmaceutically acceptable excipients.

3. The pharmaceutical composition according to claim 1, wherein the allergen is selected from the group consisting of tree pollen, weed pollen, grass pollen, epidermal, and inhalants (dog, cat, horse, guinea pig), insects, mite, food, mold, and venoms, and/or combinations thereof.

4. The pharmaceutical composition according to claim 1, wherein the allergen is selected from the group consisting of Phleum pretense, Lolium perenne, Ambrosia artemisiifolia, Periplaneta americana, Dermatophagoides farina, Apis mellifera, Peanut Allergen, and Aspergillus niger.

5. The pharmaceutical composition according to claim 1, wherein the pharmaceutical composition for oral administration is selected from the group consisting of powder, granules, pellets, beads, tablets, and capsules dosage form.

6. The pharmaceutical composition according to claim 1, wherein the pharmaceutical composition further comprises of one or more pharmaceutically acceptable excipients selected from the group consisting of diluent, binder, disintegrant, lubricant, solubilizer, glidant, surfactant, co-surfactant, an oil phase, solid carrier, seal coating polymer, enteric coating polymer, preservative, antioxidant, stabilizer, viscosity modifier, pH modifier, gelling agent, vehicle, cryoprotectant, and lyoprotectant.

7. The pharmaceutical composition according to claim 1, wherein the surfactant is selected from the group consisting of polyoxyethylene-sorbitan-fatty acid esters, polyoxyethylene products of hydrogenated vegetable oils, polyethoxylated castor oils or polyethoxylated hydrogenated castor oil, polyoxyethylene castor oil derivatives, polyethylene glycol caprylic/capric glycerides, polyethylene glycol glycerides or a combination thereof.

8. The pharmaceutical composition according to claim 1, wherein the co-surfactant is selected from the group consisting of propylene glycol, diethylene glycol monoethyl ether, propylene glycol monocaprylate, glyceryl caprylate, ethanol, glyceryl caprylate, or a combination thereof.

9. The pharmaceutical composition according to claim 1, wherein the oil phase is selected from the group consisting of propylene glycol esters, long chain fatty acids, edible oils, medium chain mono-, di-, or triglycerides, or a mixture thereof.

10. The pharmaceutical composition according to claim 1, wherein the diluent is selected from the group consisting of microcrystalline cellulose, lactose, mannitol or a combination thereof.

11. The pharmaceutical composition according to claim 1, wherein the disintegrant is selected from the group consisting of crospovidone, croscarmellose sodium, sodium starch glycolate or a combination thereof.

12. The pharmaceutical composition according to claim 1, wherein the glidant is selected from the group consisting of colloidal silicon di-oxide, starch, talc or a combination thereof.

13. The pharmaceutical composition according to claim 1, wherein the amount of allergen is present in an amount ranging from about 0.001% to 90% by total weight of the composition.

14. The pharmaceutical composition according to claim 1, wherein the composition is self-emulsifying in the gastrointestinal tract of a patient ingesting the composition and releases at a pH range of about 4 to about 7.5.

15. The pharmaceutical composition according to claim 1, wherein the composition is substantially free from hazardous amounts of allergen residues.

16. The pharmaceutical composition according to claim 1, wherein the composition forms an in situ emulsion upon contact with a biological fluid.

17. A process of preparing a self-emulsifying solid oral pharmaceutical composition comprising:
a) mixing at least one allergen to an oil phase, surfactant, and co-surfactant to form a homogenous dispersion;
b) adsorbing the homogenous dispersion obtained from step a) over a suitable solid carrier;
c) encapsulating the adsorbed mixture obtained from step b) into a capsule or compressing into a tablet.

18. A process of preparing self-emulsifying solid oral pharmaceutical composition comprising:
a) mixing at least one allergen, an oil phase, surfactant, and/or co-surfactant to prepare a core;
b) adsorbing the prepared core from step a) over a suitable solid carrier;
c) applying a seal coating layer over the core;
d) applying an enteric coating layer over the seal coating;
e) optionally coating with an outer seal coating layer over the enteric coating layer; and
f) encapsulating into a capsule or compressing into a tablet.

19. A method of treating an allergy comprising administering the composition of claim 1.

20. The method of claim 18, wherein the allergy comprises a drug allergy, a food allergy, an insect allergy, a latex allergy, a mold allergy, a pet allergy, and a pollen allergy.
